(19) United States
(12) Patent Application Publication
(10) Pub. No.: US 2023/0098222 A1
(43) Pub. Date: Mar. 30, 2023

(54) EVALUATION METHOD AND EVALUATION SYSTEM FOR SEPARATOR FOR BATTERY, PRODUCTION METHOD FOR SEPARATOR FOR BATTERY, PRODUCTION METHOD FOR ELECTRODE UNIT, AND PRODUCTION METHOD FOR BATTERY

(71) Applicant: TOYOTA JIDOSHA KABUSHIKI KAISHA, Toyota-shi (JP)

(72) Inventor: Kohei MATSUNOBU, Toyota-shi (JP)

(73) Assignee: TOYOTA JIDOSHA KABUSHIKI KAISHA, Toyota-shi (JP)

(21) Appl. No.: 17/943,318

(22) Filed: Sep. 13, 2022

(30) Foreign Application Priority Data
Sep. 24, 2021 (JP) 2021-155447

(51) Int. Cl. G01R 31/36

(52) U.S. Cl. CPC G01R 31/3644; H01M 50/489

(57) ABSTRACT

A separator is placed on a surface of a substrate to prepare a test piece. A puncturing tool is stuck into the separator, from a side of the test piece opposite to where the substrate is placed, in a thickness direction of the separator. An electrical resistance between the puncturing tool and the substrate is measured. The separator is evaluated based on a magnitude of a load applied to the puncturing tool at the time when the electrical resistance has decreased to a predetermined value. Each of the substrate and the puncturing tool is electrically conductive.

This nonprovisional application is based on Japanese Patent Application No. 2021-155447 filed on Sep. 24, 2021, with the Japan Patent Office, the entire contents of which are hereby incorporated by reference.

### BACKGROUND

### Field

The present disclosure relates to an evaluation method and an evaluation system for a separator for a battery, a production method for a separator for a battery, a production method for an electrode unit, and a production method for a battery.

### Description of the Background Art

Japanese Patent Laying-Open No. 2014-32173 discloses an apparatus for measuring puncture strength.

### SUMMARY

Hereinafter, “a separator”, “an electrode”, and “an electrode assembly” refer to “a separator for a battery”, “an electrode for a battery”, and “an electrode assembly for a battery”, respectively, unless otherwise specified. Moreover, “an electrode” may be used as a generic name for “a positive electrode” and “a negative electrode”.

A separator is in film form. Inside a battery, a separator electrically insulates a positive electrode from a negative electrode. For example, during production of a battery, a foreign object (such as a metal fragment) may be trapped between a separator and an electrode. Inside the battery, the foreign object may apply a local load to the separator. The separator may be deformed locally, potentially resulting in insulation resistance decrease, voltage failure, and/or the like.

Conventionally, as an evaluation method for a separator, “puncture strength test” described in “JIS Z 1707 General rules of plastic films for food packaging” is widely used.

FIG. 1 is a view describing a puncture strength test.

A test fragment 1 (a film) is placed on a hole-punched stage 2. Hole-punched stage 2 has a hole 3 in it. Test fragment 1 is positioned above hole 3. A needle 4 has a hemispherical tip. Needle 4 has a diameter of 1.0 mm and a tip radius of 0.5 mm. Needle 4 is stuck into test fragment 1. The test velocity is 50±5 mm/min. A maximum force exerted until needle 4 penetrates through test fragment 1 is measured. This maximum force (maximum load applied to needle 4) is regarded as the puncture strength [unit, N] of test fragment 1. The greater the puncture strength is, the more resistant to local deformation and the less likely to break the film is considered to be.

In a conventional puncture strength test, test fragment 1 can extend in the direction in which needle 4 moves (in the Z-axis direction). Because of this, a film that extends easily tends to have a greater puncture strength. Inside an actual battery, there would be little space for a separator to extend into. Therefore, puncture strength is not considered to be a suitable index for the strength of a separator inside an actual battery.

In some instances, an electrode unit may be produced by, for example, bonding a separator to an electrode. For example, an electrically insulating material may be applied to a surface of an electrode to form a separator that is bonded to the electrode. It is difficult to evaluate the strength of the separator included in the electrode unit. That is, it is difficult to distinguish the strength of the separator from the strength of the underlying material (the electrode). There has been a demand for an evaluation method that is applicable to a separator included in an electrode unit.

An object of the present disclosure is to provide an evaluation method for a separator for a battery.

Hereinafter, the technical configuration and effects of the present disclosure will be described. It should be noted that the action mechanism according to the present specification includes presumption. The action mechanism does not limit the technical scope of the present disclosure.

1. An evaluation method for a separator for a battery includes the following (a) to (d):
(a) preparing a test piece by placing a separator on a surface of a substrate;
(b) sticking a puncturing tool into the separator, from a side of the test piece opposite to where the substrate is placed, in a thickness direction of the separator;
(c) measuring an electrical resistance between the puncturing tool and the substrate while the puncturing tool is being stuck into the separator; and
(d) evaluating the separator based on a magnitude of a load applied to the puncturing tool at the time when the electrical resistance has decreased to a predetermined value, wherein
each of the substrate and the puncturing tool is electrically conductive.

Within the test piece according to the present disclosure, the separator is supported by the substrate. For example, the substrate may be a simulant electrode. For example, the substrate may be an actual electrode. For example, the separator may be simply placed on the substrate. For example, the separator may be bonded to the substrate.

In the evaluation method according to the present disclosure, the puncturing tool is stuck into the front face of the separator while the back face of the separator is being supported by the substrate. That is, the puncturing tool is stuck into the separator while extension of the separator is hindered. In this way, it is possible to simulate the deformation behavior of a separator inside an actual battery.

The puncturing tool is electrically conductive. The puncturing tool may be a metal needle and/or the like, for example. The puncturing tool moves in the thickness direction of the separator. While the puncturing tool is moving, the electrical resistance between the puncturing tool and the substrate is monitored. The electrical resistance is considered to be corresponding to the insulation resistance at the time when a foreign object enters between the separator and the electrode. As the puncturing tool moves, the electrical resistance decreases.

As the puncturing tool moves, the load applied to the puncturing tool increases. In the present disclosure, the load [unit, N] applied at the time when the electrical resistance has decreased to a predetermined value is measured. Hereinafter, the predetermined value of electrical resistance is also called “a short circuit resistance”. The load at a short circuit resistance is also called “a short circuit load”. The short circuit resistance may be determined by referring to an insulation resistance required between electrodes within a battery, for example. The short circuit load may be used to evaluate if the separator can maintain electric insulation without being broken when, for example, a foreign object enters between electrodes. The short circuit load may serve as a useful index in designing, development, and production of a separator.

The short circuit load according to the present disclosure may be measured before the puncturing tool penetrates through the separator. Further, the test piece corresponds to an electrode unit that includes a separator bonded to an electrode. Therefore, it is possible to evaluate the strength of a separator included in an electrode unit.

2. The evaluation method for a separator for a battery may further include, for example, the following (e):
(e) evaluating the separator based on an amount of displacement of the puncturing tool at the time when the electrical resistance has decreased to the predetermined value.

Hereinafter, the amount of displacement is also called “an amount of short circuit displacement”. From the amount of displacement of the puncturing tool and the initial thickness of the separator, the amount of separator crushing may be derived. The amount of short circuit displacement is regarded as corresponding to the maximum amount of separator crushing at which the separator can maintain electric insulation inside the battery. The amount of short circuit displacement may also serve as a useful index in designing, development, and production of a separator.

3. The substrate may include an electrode for a battery, for example.

Depending on the mechanical characteristics of the electrode (such as the hardness, for example), the load applied to a foreign object and to a separator inside an actual battery may change. With the substrate being an actual electrode, the environment inside an actual battery is expected to be simulated even better.

4. The substrate may include an electrode assembly for a battery, for example. The electrode assembly for a battery includes a plurality of the electrodes for a battery.

Generally, a battery includes an electrode assembly. The electrode assembly is a group of electrodes. Depending on the structure and mechanical characteristics of the electrode assembly, the load applied to a foreign object and to a separator inside an actual battery may change. With the substrate being an actual electrode assembly, the environment inside an actual battery is expected to be simulated even better.

5. An evaluation system includes a stage, a driver apparatus, a resistivity-measuring apparatus, and a load-measuring apparatus.

The stage is configured to receive the test piece on itself.

The driver apparatus is configured to move the puncturing tool in the thickness direction of the separator toward the test piece on the stage.

The resistivity-measuring apparatus is configured to measure an electrical resistance between the puncturing tool and the substrate.

The load-measuring apparatus is configured to measure the load applied to the puncturing tool.

In the evaluation system according to the above “5”, the evaluation method for a separator for a battery according to the above “1” may be implemented.

6. The evaluation system may further include a displacement-measuring apparatus, for example

The displacement-measuring apparatus is configured to measure an amount of displacement of the puncturing tool.

In the evaluation system according to the above “6”, the evaluation method for a separator for a battery according to the above “2” may be implemented.

7. The stage may be electrically conductive. The resistivity-measuring apparatus may be configured to measure an electrical resistance between the puncturing tool and the stage.

When the substrate and the stage are electrically connected with each other, it is considered that the electrical resistance between the puncturing tool and the stage includes the electrical resistance between the puncturing tool and the substrate. Measuring the electrical resistance between the puncturing tool and the stage enables indirect measurement of the electrical resistance between the puncturing tool and the substrate. Depending on the shape of the test piece (the substrate), it may be easier and more simple to measure the electrical resistance between the puncturing tool and the stage.

8. A production method for a separator for a battery includes the following (A1) and (A2):

(A1) producing a separator; and

(A2) evaluating the separator by the evaluation method for a separator for a battery.

The evaluation method for a separator for a battery may be applied to, for example, designing and development of a separator. For example, based on the magnitude of short circuit load, separator designing may be attempted. The evaluation method for a separator for a battery may be applied to, for example, quality control of a separator. For example, during separator production, sampling inspection may be carried out by the evaluation method for a separator for a battery. For example, the magnitude of short circuit load may be used to assess whether a production lot is good or not.

9. A production method for an electrode unit includes the following (B1) and (B2):

(B1) producing an electrode unit by placing a separator on a surface of an electrode for a battery; and

(B2) evaluating the separator by the evaluation method for a separator for a battery, by using the electrode unit as a test piece.

Within the electrode unit, the separator is bonded the electrode. In the evaluation method for a separator for a battery, the electrode unit may serve as a test piece. In the evaluation method for a separator for a battery, even when the separator is bonded to the electrode, the strength of the separator can be independently evaluated.

The evaluation method for a separator for a battery may be applied to, for example, designing and development of an electrode unit. For example, based on the magnitude of short circuit load, separator designing may be attempted. For example, based on the magnitude of short circuit load, electrode designing may be attempted. The evaluation method for a separator for a battery may be applied to, for example, quality control of an electrode unit. For example, during electrode unit production, sampling inspection may be carried out by the evaluation method for a separator for a battery. For example, the magnitude of short circuit load may be used to assess whether a production lot is good or not.

10. The separator may be bonded to the surface of the electrode for a battery.

When the separator is bonded to the electrode, it is difficult to remove the separator from the electrode. Moreover, removing the separator from the electrode may break the separator. When the separator is broken, the strength of the separator may not be properly evaluated. In the evaluation method for a separator for a battery, separator evaluation can be performed while the separator is bonded to the electrode.

11. A production method for a battery includes the following (C1) and (C2):

(C1) producing an electrode unit by the production method for an electrode unit; and

(C2) producing a battery including the electrode unit.

For example, a plurality of electrode units may be stacked to form an electrode assembly. The electrode assembly may be placed in a casing to produce a battery. The production method for an electrode unit includes the evaluation method for a separator for a battery. The battery is expected to have a short circuit resistance the level of which depends on the short circuit load of the separator.

12. A production method for a battery includes the following (D1) and (D2):

(D1) evaluating a separator by the evaluation method for a separator for a battery; and

(D2) producing a battery including the separator.

The evaluation method for a separator for a battery may be applied to, for example, designing and development of a battery. For example, the magnitude of short circuit load of the separator may be adjusted so as to suit the battery specifications. The evaluation method for a separator for a battery may be applied to, for example, quality control of a battery. For example, a battery may be produced by using a separator that has a short circuit load equal to or more than a reference value.

The foregoing and other objects, features, aspects and advantages of the present disclosure will become more apparent from the following detailed description of the present disclosure when taken in conjunction with the accompanying drawings.

### BRIEF DESCRIPTION OF THE DRAWINGS

FIG. 1 is a view describing a puncture strength test.

FIG. 2 is a conceptual view illustrating an evaluation system according to the present embodiment.

FIG. 3 is a schematic flowchart of an evaluation method for a separator for a battery according to the present embodiment.

FIG. 4 is a conceptual view illustrating an example of a test piece.

FIG. 5 is a schematic flowchart of a first production method.

FIG. 6 is a schematic flowchart of a second production method.

FIG. 7 is a schematic flowchart of a third production method.

FIG. 8 is a graph for the relationship between puncture strength and separator thickness in a first evaluation example.

FIG. 9 is a conceptual view illustrating a second evaluation example.

FIG. 10 is a conceptual view illustrating a third evaluation example.

FIG. 11 is a schematic view illustrating a puncturing tool in a fourth evaluation example.

FIG. 12 is a graph for the relationship between short circuit load and separator thickness in a fourth evaluation example.

FIG. 13 is a schematic view illustrating a puncturing tool in a fifth evaluation example.

FIG. 14 is a graph for the relationship between short circuit load and separator thickness in a fifth evaluation example.

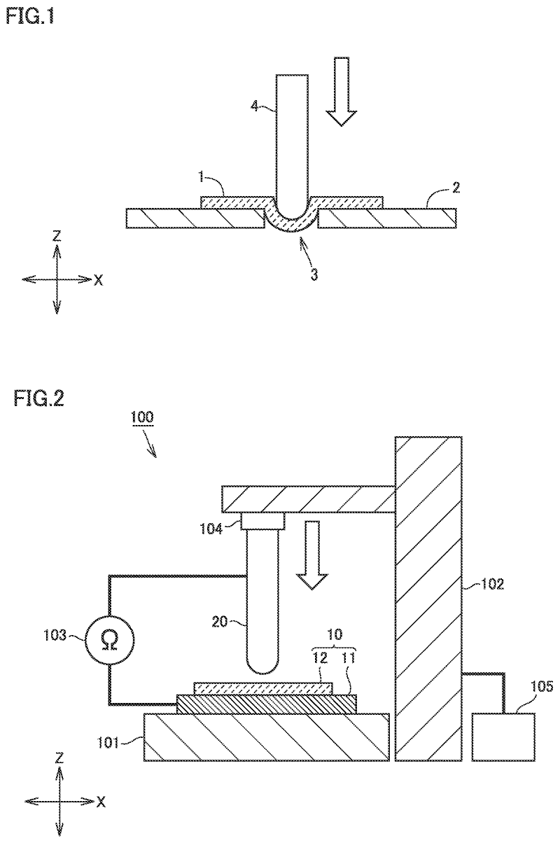

### DESCRIPTION OF THE PREFERRED EMBODIMENTS

### <Definitions of Terms, etc.>

Next, an embodiment of the present disclosure (which may also be simply called “the present embodiment”) and an example of the present disclosure (which may also be simply called “the present example”) will be described. It should be noted that neither the present embodiment nor the present example limits the technical scope of the present disclosure.

Herein, expressions such as “comprise”, “include”, and “have”, and other similar expressions (such as “be composed of”, for example) are open-ended expressions. In an open-ended expression, in addition to an essential component, an additional component may or may not be further included. The expression “consist of” is a closed-end expression. However, even when a closed-end expression is used, impurities present under ordinary circumstances as well as an additional element irrelevant to the technique according to the present disclosure are not excluded. The expression “consist essentially of” is a semiclosed-end expression. A semiclosed-end expression tolerates addition of an element that does not substantially affect the fundamental, novel features of the technique according to the present disclosure.

In the method described in the present specification, the order for implementing a plurality of steps, operations, processes, and the like is not limited to the described order, unless otherwise specified. For example, a plurality of steps may proceed simultaneously. For example, a plurality of steps may be implemented in reverse order.

Herein, expressions such as “may” and “can” are not intended to mean “must” (obligation) but rather mean “there is a possibility” (tolerance).

Herein, any geometric term (such as “parallel”, “vertical”, and “perpendicular”, for example) should not be interpreted solely in its exact meaning. For example, “parallel” may mean a geometric state that is deviated, to some extent, from exact “parallel”. Any geometric term herein may include tolerances and/or errors in terms of design, operation, production, and/or the like. The dimensional relationship in each figure may not necessarily coincide with the actual dimensional relationship. The dimensional relationship (in length, width, thickness, and the like) in each figure may have been changed for the purpose of assisting the understanding of the technique according to the present disclosure. Further, a part of a configuration may have been omitted.

Herein, a numerical range such as “from m to n %” includes both the upper limit and the lower limit, unless otherwise specified. That is, “from m to n %” means a numerical range of “not less than m % and not more than n %”. Moreover, “not less than m % and not more than n %” includes “more than m % and less than n %”. Further, any numerical value selected from a certain numerical range may be used as a new upper limit or a new lower limit. For example, any numerical value from a certain numerical range may be combined with any numerical value described in another location of the present specification or in a table or a drawing to set a new numerical range.

Herein, all the numerical values are regarded as being modified by the term “about”. The term “about” may mean ±5%, ±3%, ±1%, and/or the like, for example. Each numerical value may be an approximate value that can vary depending on the implementation configuration of the technique according to the present disclosure.

Each numerical value may be expressed in significant figures. Each measured value may be the average value obtained from multiple measurements performed. The number of measurements may be 3 or more, or may be 5 or more, or may be 10 or more. Generally, the greater the number of measurements is, the more reliable the average value is expected to be. Each measured value may be rounded off based on the number of the significant figures. Each measured value may include an error occurring due to an identification limit of the measurement apparatus, for example.

Herein, “electrically conductive” means that at least part of an object in question has an electric resistivity equal to or less than 10⁷ Ω·cm. The entire object in question may have an electric resistivity equal to or less than 10⁷ Ω·cm, or a part of the object in question may have an electric resistivity equal to or less than 10⁷ Ω·cm. For example, when the object in question is a single-ingredient material such as a metal foil, the entire object in question may have an electric resistivity equal to or less than 10⁷ Ω·cm. For example, when the object in question is a composite material such as an electrode or an electrode assembly, a part of the object in question may have an electric resistivity equal to or less than 10⁷ Ω·cm.

Herein, “air permeability” refers to the “air resistance” defined by “JIS P 8117 Paper and board-Determination of air permeance and air resistance (medium range)-Gurley method”. The air permeability is measured by a Gurley test method.

The present embodiment may be applied to any separator for a battery. For example, the present embodiment may be applied to a separator for a lithium-ion battery.

### <Evaluation System>

FIG. 2 is a conceptual view illustrating an evaluation system according to the present embodiment.

Hereinafter, “an evaluation system according to the present embodiment” may be simply called “a present evaluation system”.

A present evaluation system 100 may be used for evaluating the strength of a separator. Present evaluation system 100 includes a stage 101, a driver apparatus 102, a resistivity-measuring apparatus 103, and a load-measuring apparatus 104. Present evaluation system 100 may further include a displacement-measuring apparatus 105, and/or the like, for example.

Present evaluation system 100 may further include a control apparatus, a computing apparatus, a recording apparatus, a display apparatus (none of these is illustrated), and/or the like, for example.

For example, each of these apparatuses may be independent of each other. For example, some of or all of these apparatuses may be integrated into a single member. For example, present evaluation system 100 may include a texture analyzer, a precision universal tester (an autograph), and/or the like. For example, the texture analyzer may include stage 101, driver apparatus 102, load-measuring apparatus 104, and displacement-measuring apparatus 105.

### <<Stage>>

Stage 101 is configured to receive a test piece 10 on itself. Test piece 10 includes a substrate 11 and a separator 12. The details of test piece 10 will be described below. For example, stage 101 may comprise a jig for securing test piece 10. For example, the surface of stage 101 may be flat. “Flat” means that there is substantially no hole, irregularities, or the like. Stage 101 may be formed of any material. For example, stage 101 may be electrically insulating. For example, stage 101 may include a resin plate and/or the like. For example, stage 101 may be electrically conductive. For example, stage 101 may include a metal plate and/or the like. For example, stage 101 may be made of stainless steel (SUS), or may be made of aluminum (Al) alloy, and/or the like.

### <<Driver Apparatus>>

Driver apparatus 102 is configured to move a puncturing tool 20 in a thickness direction of separator 12 toward test piece 10 on stage 101. Driver apparatus 102 is capable of moving puncturing tool 20 by any drive principle. Driver apparatus 102 may include a servomotor, a ball screw, a crosshead, and/or the like, for example. Driver apparatus 102 may be configured to move puncturing tool 20 in, for example, a direction vertical to the surface of stage 101 (in the Z-axis direction in FIG. 2). Driver apparatus 102 may be configured to move puncturing tool 20 at a sufficiently low velocity. Driver apparatus 102 may be configured to move puncturing tool 20 at a test velocity from 0.01 to 100 mm/min, for example.

### <<Puncturing Tool>>

Puncturing tool 20 is attached to driver apparatus 102. Puncturing tool 20 may be detachable, for example. Puncturing tool 20 may be in needle shape, for example. Puncturing tool 20 is electrically conductive. For example, puncturing tool 20 may be made of iron (Fe), or may be made of SUS, and/or the like. As puncturing tool 20, a needle used in a conventional puncture strength test may be used, for example. The size and the tip shape of puncturing tool 20 may be selected as appropriate depending on, for example, an expected foreign object, the mode of failure, and/or the like. Puncturing tool 20 may have a diameter from 0.1 to 10 mm, for example. The diameter refers to the maximum diameter of the barrel (which is the part excluding the tip). For example, the tip shape of puncturing tool 20 may be hemispherical. For example, the tip shape of puncturing tool 20 may be taper R. For example, the taper angle may be from 30 to 90°. For example, the tip radius may be from 0.01 to 1 mm. For example, the sharper the tip shape is, the harsher the evaluation conditions seem to be.

### <<Resistivity-Measuring Apparatus>>

Resistivity-measuring apparatus 103 is configured to measure the electrical resistance between puncturing tool 20 and substrate 11. Resistivity-measuring apparatus 103 may include a commercially available tester, insulation resistance meter, and/or the like, for example. The measurement range of resistivity-measuring apparatus 103 may be selected as appropriate depending on, for example, an expected foreign object, the mode of failure, and/or the like. The upper limit to the measurement range may be from 0.1 to 100 MΩ, or may be from 10 to 50 MΩ, for example.

Resistivity-measuring apparatus 103 may be connected to puncturing tool 20 and substrate 11 via a lead wire, a clip, and/or the like, for example. The electrical resistance between puncturing tool 20 and stage 101 may be measured when stage 101 is electrically conductive and stage 101 is electrically connected with substrate 11. Depending on the shape of test piece 10 (substrate 11), it may be easier and more simple to measure the electrical resistance between puncturing tool 20 and stage 101.

### <<Load-Measuring Apparatus>>

Load-measuring apparatus 104 is configured to measure the load applied to puncturing tool 20. Load-measuring apparatus 104 may include a load cell and/or the like, for example. The measurement range and measurement precision of load-measuring apparatus 104 may be selected as appropriate depending on the strength and/or thickness of separator 12, for example. The measurement range of load-measuring apparatus 104 may be from 0.1 to 1000 N, for example.

### <<Displacement-Measuring Apparatus>>

Present evaluation system 100 may further include displacement-measuring apparatus 105. Displacement-measuring apparatus 105 is configured to measure an amount of displacement of puncturing tool 20. Displacement-measuring apparatus 105 is capable of measuring the amount of displacement of puncturing tool 20 by any method. For example, displacement-measuring apparatus 105 may compute the amount of displacement using the moving velocity (test velocity) and the moving time of puncturing tool 20.

### <<Display Apparatus>>

Present evaluation system 100 may further include a display apparatus (not illustrated), for example. The display apparatus may include a liquid crystal panel and/or the like, for example. The display apparatus may be configured to display, for example, at least one selected from the group consisting of test velocity, load (test force), amount of displacement, and electrical resistance.

### <<Recording Apparatus>>

Present evaluation system 100 may further include a recording apparatus (not illustrated), for example. The recording apparatus may include a data logger and/or the like, for example. The recording apparatus may be configured to record at least one selected from the group consisting of electrical resistance, load, and amount of displacement. The recording apparatus may record the time course of a target value (such as a load and/or an amount of displacement, for example).

### <<Control Apparatus>>

Present evaluation system 100 may further include a control apparatus (not illustrated), for example. The control apparatus may control operation of each apparatus, coordination between apparatuses, and/or the like, for example. The control apparatus may have a computing function, for example. The control apparatus may be configured, for example, to acquire the time course of electrical resistance and load from the recording apparatus and compute the load at a predetermined electrical resistance. The control apparatus may compute the amount of displacement of puncturing tool 20.

### <Evaluation Method>

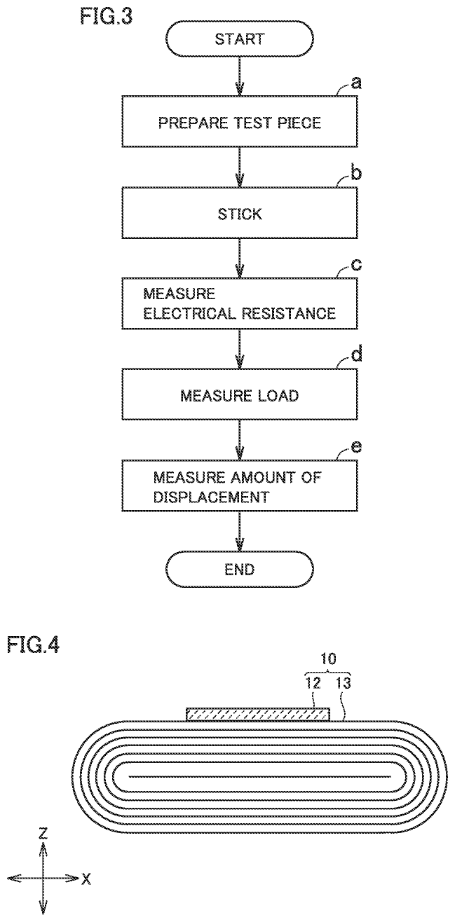

FIG. 3 is a schematic flowchart of an evaluation method for a separator for a battery according to the present embodiment. Hereinafter, “the evaluation method for a separator for a battery according to the present embodiment” may be simply called “the present evaluation method”. The present evaluation method may be implemented on present evaluation system 100. The present evaluation method includes “(a) preparing a test piece”, “(b) sticking”, “(c) measuring an electrical resistance”, and “(d) measuring a load”. The present evaluation method may further include “(e) measuring an amount of displacement”. The order of (a) to (e) given in FIG. 3 is assigned merely for the sake of convenience. For example, (b) to (e) may be implemented substantially at the same time.

### <<(a) Preparing Test Piece>>

The present evaluation method includes preparing test piece 10 (a test work) by placing separator 12 on a surface of substrate 11 (see FIG. 2).

For example, depending on the size and/or the like of stage 101, separator 12 is cut into a predetermined size. For example, separator 12 may be simply placed on substrate 11 to prepare test piece 10. For example, separator 12 may be bonded to a surface of substrate 11 to prepare test piece 10.

For example, an electrode unit may be produced. The electrode unit includes an electrode and separator 12. Separator 12 is bonded to the surface of the electrode. The electrode unit may be cut into a predetermined size to prepare test piece 10. In this case, the electrode is regarded as substrate 11.

### <Separator>

Separator 12 is in film shape. Separator 12 may have a thickness from 10 to 50 μm, or may have a thickness from 10 to 20 μm, for example. Separator 12 is electrically insulating. Separator 12 may include resin, ceramic, and/or the like, for example.

Separator 12 may be porous, for example. Separator 12 may have an air permeability from 100 to 500 s/mL, for example. Separator 12 may be for a liquid-type battery, for example. The liquid-type battery includes an electrolyte solution. The electrolyte solution is capable of permeating into pores of separator 12.

For example, separator 12 may include polyolefin and/or the like. For example, separator 12 may include polyethylene (PE), polypropylene (PP), and/or the like. Separator 12 may have a monolayer structure. For example, separator 12 may consist essentially of a PE layer. For example, separator 12 may have a multilayer structure. For example, separator 12 may include a three-layer structure. For example, a PP layer, a PE layer, and a PP layer may be stacked in this order to form a three-layer structure.

For example, separator 12 may be a composite material of resin and ceramic. For example, the ceramic may be in particle form. For example, ceramic, a binder, and a dispersion medium may be mixed to prepare a slurry. The slurry may be applied to a surface of a resin film to form a ceramic layer. The ceramic may include alumina, boehmite, titania, zirconia, silica, and/or the like, for example. The binder may include polyvinylidene difluoride (PVdF) and/or the like, for example.

Separator 12 may be produced by any method. Separator 12 may be produced by a dry process, or may be produced by a wet process. For example, separator 12 may be produced by stretching, phase separation, and/or the like. For example, separator 12 may be “a self-standing film”. The self-standing film refers to a film that is capable of maintaining its shape by itself. For example, separator 12 may be “a non-self-standing film”. The non-self-standing film refers to a film that requires a support for maintaining its shape. For example, a particle-form resin, a particle-form ceramic, and/or the like may be applied to a surface of the electrode to form a non-self-standing film on the surface of the electrode.

For example, separator 12 may be non-porous. For example, separator 12 may be for an all-solid-state battery. For example, a solid electrolyte may be in particle form. For example, a solid electrolyte, a binder, and a dispersion medium may be mixed to prepare a slurry. The slurry may be applied to a surface of the electrode to form separator 12 (a solid electrolyte layer). The solid electrolyte layer may be compressed to closely pack the solid electrolyte layer. The solid electrolyte may include Li₂S-P₂S₅ and/or the like, for example.

### <Substrate>

For example, substrate 11 may be in sheet form, in plate form, and/or the like. For example, the thickness of substrate 11 may be determined so that separator 12 has a moderate margin for crushing. For example, substrate 11 may have a thickness of 1 μm or more, or may have a thickness of 10 μm or more, or may have a thickness of 100 μm or more. Substrate 11 is electrically conductive. For example, substrate 11 may include a metal foil, a metal plate, and/or the like. For example, substrate 11 may include a copper (Cu) foil, an Al foil, and/or the like.

For example, substrate 11 may include an electrode. When substrate 11 is an electrode, the environment inside an actual battery is expected to be simulated even better. The electrode may be a positive electrode, or may be a negative electrode. For example, in a lithium-ion battery, a negative electrode tends to be softer than a positive electrode. When a foreign object enters into a lithium-ion battery, the foreign object tends to be trapped in the negative electrode side. With substrate 11 being a negative electrode, for example, foreign object contamination in a lithium-ion battery may be easily simulated.

The electrode may include an active material layer and a current collector, for example. The active material layer is placed on a surface of the current collector. The active material layer may be placed on only one side of the current collector, or may be placed on both sides of the current collector.

For example, the current collector may have a thickness from 5 to 50 μm, or may have a thickness from 5 to 20 μm. For example, the current collector may include a metal foil and/or the like. For example, the current collector may include a Cu foil, a Cu alloy foil, an Al foil, an Al alloy foil, a nickel (Ni) foil, a Ni alloy foil, a titanium (Ti) foil, a Ti alloy foil, and/or the like.

The active material layer may have a thickness from 10 to 200 μm, for example. The active material layer includes a positive electrode active material or a negative electrode active material. The positive electrode active material may include lithium nickel cobalt manganese oxide, lithium nickel cobalt aluminate, lithium iron phosphate, and/or the like, for example. The negative electrode active material may include graphite, silicon, silicon oxide, tin, tin oxide, lithium titanium oxide, metal lithium, and/or the like, for example. The active material layer may further include a conductive material, a binder, and/or the like. The conductive material may include carbon black and/or the like, for example. The amount of the conductive material to be used may be, for example, from 0.1 to 10 parts by mass relative to 100 parts by mass of the active material. The binder may include PVdF, carboxymethylcellulose (CMC), styrene-butadiene rubber (SBR), and/or the like, for example. The amount of the binder to be used may be, for example, from 0.1 to 10 parts by mass relative to 100 parts by mass of the active material.

FIG. 4 is a conceptual view illustrating an example of a test piece.

Substrate 11 may include an electrode assembly 13, for example. Electrode assembly 13 includes a plurality of electrodes. Electrode assembly 13 may have any form. Electrode assembly 13 may be a wound-type one, or may be a stack-type one, for example. With substrate 11 being electrode assembly 13, the environment inside an actual battery is expected to be simulated even better.

### <<(b) Sticking>>

The present evaluation method includes sticking puncturing tool 20 into separator 12, from a side of test piece 10 opposite to where substrate 11 is placed, in a thickness direction of separator 12.

For example, present evaluation system 100 and puncturing tool 20 are prepared (see FIG. 2). The details of present evaluation system 100 and puncturing tool 20 are as described above. Puncturing tool 20 is attached to driver apparatus 102. To puncturing tool 20, resistivity-measuring apparatus 103 is connected. To substrate 11 (or stage 101), resistivity-measuring apparatus 103 is connected. The electrical resistance between puncturing tool 20 and substrate 11 is measured. At this point in time, the electrical resistance may exceed the upper limit to the measurement range of resistivity-measuring apparatus 103. That is, the displayed value of the electrical resistance may be infinity, for example.

For example, at any test velocity, puncturing tool 20 may be lowered to reach immediately above separator 12. It seems that before puncturing tool 20 comes into contact with separator 12, the test velocity does not affect the results of evaluation. By moving puncturing tool 20 to immediately above separator 12 at a relatively high test velocity, it is possible to reduce testing time.

Then, the test velocity (the sticking velocity) is set. Puncturing tool 20 is stuck into separator 12 at a substantially constant test velocity. The test velocity may be adjusted as appropriate depending on the thickness of separator 12, the sampling frequency for load and electrical resistance, and/or the like. When the test velocity is too fast relative to the thickness of separator 12 and the sampling frequency, for example, it may be difficult to acquire the load applied at the time of a short circuit. The test velocity may be from 0.001 to 10 mm/min, or may be from 0.01 to 1 mm/min, or may be from 0.1 to 0.5 mm/min, for example.

The direction in which puncturing tool 20 moves (a sticking direction) may be parallel to the thickness direction of separator 12. In the present evaluation method, extension of separator 12 in the sticking direction seems to be hindered. This is because the back face of separator 12 is supported by substrate 11. With the extension of separator 12 being hindered, the environment inside an actual battery is expected to be simulated.

### <<(c) Measuring Electrical Resistance>>

The present evaluation method includes measuring an electrical resistance between puncturing tool 20 and substrate 11 while puncturing tool 20 is being stuck into separator 12. The electrical resistance may be measured with resistivity-measuring apparatus 103, for example (see FIG. 2).

### <<(d) Measuring Load>>

The present evaluation method includes evaluating separator 12 based on a magnitude of a short circuit load, which is a load applied to puncturing tool 20 at the time when the electrical resistance has decreased to a predetermined value (a short circuit resistance).

The load applied to puncturing tool 20 may be measured with load-measuring apparatus 104, for example (see FIG. 2). The short circuit resistance may be determined by referring to, for example, an insulation resistance required between electrodes within the battery. For example, the short circuit resistance may be the upper limit to the measurement range of resistivity-measuring apparatus 103. For example, at the time when the displayed value on resistivity-measuring apparatus 103 has changed from infinity (Go) to a numerical value, it may be judged that the electrical resistance has reached short circuit resistance. It is considered that display of a numerical value on resistivity-measuring apparatus 103 indicates a very small current flowing between puncturing tool 20 and substrate 11. In other words, it is considered that display of a numerical value on resistivity-measuring apparatus 103 indicates an occurrence of a micro-short-circuit. The short circuit resistance may be set at from 0.1 to 100 MΩ, or may be set at from 1 to 100 MΩ, or may be set at from 5 to 50 MΩ, or may be set at from 30 to 50 MΩ, for example.

In the present evaluation method, the load applied at the time when the electrical resistance has reached the short circuit resistance (a short circuit load) is measured. At the time when the electrical resistance has reached the short circuit resistance, puncturing tool 20 may be stopped. After puncturing tool 20 is stopped, the short circuit load may be measured. Puncturing tool 20 may be continuously moved after the electrical resistance has reached the short circuit resistance. For example, the time course of electrical resistance and load may be used to identify the load for the short circuit resistance. The time course of electrical resistance and load may be accumulated in the recording apparatus, for example.

The short circuit load may be used to evaluate if the separator can maintain electric insulation without being broken when, for example, a foreign object enters between electrodes. For example, separator 12 may be evaluated based on the magnitude of short circuit load. For example, the greater the short circuit load is, the higher the short circuit resistance of separator 12 may be rated.

After the short circuit load is measured, movement of puncturing tool 20 and measurement of electrical resistance and load may be either continued or stopped. Puncturing tool 20 may be stopped at the time when it has penetrated through separator 12, or may be stopped before it penetrates through separator 12.

### <<(e) Measuring Amount of Displacement>>

The present evaluation method may include evaluating separator 12 based on an amount of short circuit displacement, namely, an amount of displacement of puncturing tool 20 at the time when the electrical resistance has decreased to a predetermined value (a short circuit resistance). The amount of displacement of puncturing tool 20 may be measured with displacement-measuring apparatus 105 (see FIG. 2). For example, from the amount of displacement of puncturing tool 20 and the initial thickness of separator 12, the amount of crushing of separator 12 may be derived. By this, it is possible to evaluate the amount of crushing of separator 12 inside the battery required for causing a short circuit, for example.

### <First Production Method>

FIG. 5 is a schematic flowchart of a first production method.

The first production method is a production method for a separator. The first production method includes “(A1) producing a separator” and “(A2) evaluating the separator”.

### <<(A1) Producing Separator>>

The first production method includes producing separator 12. In the first production method, separator 12 is a self-standing film. Separator 12 may be produced by any method. For example, a product for mass manufacturing may be produced. For example, a prototype may be produced.

### <<(A2) Evaluating Separator>>

The first production method includes evaluating the separator by the present evaluation method.

The present evaluation method may be applied to, for example, designing and development of separator 12. For example, a short circuit load of a prototype separator 12 may be measured. Separator 12 may be modified so as to increase the short circuit load.

The present evaluation method may be applied to, for example, quality control of separator 12. For example, during production of separator 12, sampling inspection may be carried out. The magnitude of short circuit load may be used to assess whether a production lot is good or not.

### <Second Production Method>

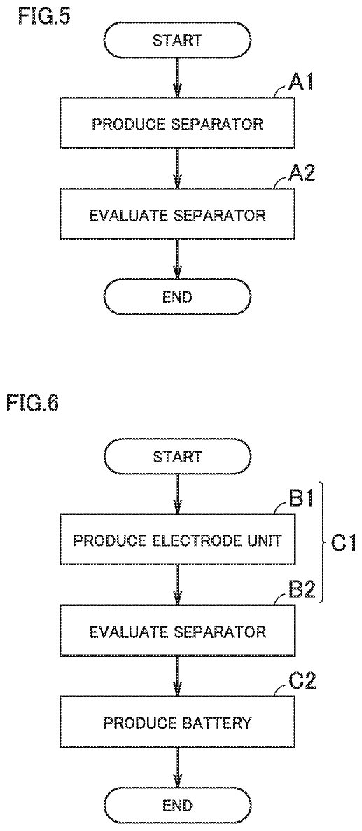

FIG. 6 is a schematic flowchart of a second production method.

The second production method includes a production method for an electrode unit. The second production method includes “(B1) producing an electrode unit” and “(B2) evaluating the separator”. The second production method also includes a production method for a battery. More specifically, the second production method may include “(C1) producing an electrode unit” and “(C2) producing a battery”.

### <<(B1) Producing Electrode Unit>>

The second production method includes producing an electrode unit by placing separator 12 on a surface of an electrode.

The electrode unit is a part for a battery. For example, the electrode units may be stacked to form an electrode assembly. Within the electrode unit, separator 12 is bonded to the electrode.

The electrode unit may be produced by any method. For example, an active material is prepared. For example, the active material may be in particle form. A current collector is prepared. For example, the current collector may include a metal foil and/or the like. For example, the active material, a binder, and a dispersion medium may be mixed to prepare a slurry. The slurry may be applied to a surface of the current collector to form an active material layer. The active material layer may be compressed to produce an electrode.

For example, a polymer solution may be applied to a surface of the electrode, for example, to form a polymer film on the surface of the electrode. For example, pores may be formed in the polymer film by a phase separation method. In this way, separator 12 that is bonded to the electrode may be formed. In other words, an electrode unit may be produced.

For example, separator 12 (a self-standing film) may be bonded to a surface of the electrode to produce an electrode unit. For example, an adhesive may be used to bond separator 12 to the electrode. The adhesive may include PVdF and/or the like, for example. For example, at least one of heat and pressure may be applied to a stack of separator 12 and the electrode to make separator 12 attached to the electrode. The entire separator 12 may be bonded to the electrode, or a part of it may be bonded to the electrode.

### <<(B2) Evaluating Separator>>

The second production method includes evaluating separator 12 by the present evaluation method by using the electrode unit as test piece 10. For example, the electrode unit may be cut into a predetermined size to prepare test piece 10. In the present evaluation method, evaluation of separator 12 can be performed while separator 12 is bonded to the electrode. The present evaluation method is suitable for evaluation of separator 12 included in an electrode unit.

The present evaluation method may be applied to, for example, designing and development of an electrode unit. For example, in a prototype electrode unit, the short circuit load of separator 12 may be measured. The electrode unit may be modified so as to increase the short circuit load.

The present evaluation method may be applied to, for example, quality control of an electrode unit. For example, during electrode unit production, sampling inspection may be carried out. The magnitude of short circuit load may be used to assess whether a production lot is good or not.

### <<(C1) Producing Electrode Unit>>

In “(C1) producing an electrode unit”, the above-described “(B1) producing an electrode unit” and “(B2) evaluating the separator” are employed to produce an electrode unit and evaluate separator 12.

### <<(C2) Producing Battery>>

The second production method includes producing a battery including the electrode unit. The battery may be produced by any method. For example, the electrode units may be stacked to form an electrode assembly. The electrode assembly and an electrolyte solution may be encapsulated inside a casing to produce a battery. For example, the casing may be a metal vessel and/or the like, or may be a pouch made of a metal-foil-laminated film, and/or the like.

The battery includes separator 12 the short circuit load of which is already evaluated. The battery may have a short circuit resistance the level of which depends on the short circuit load of separator 12.

### <Third Production Method>

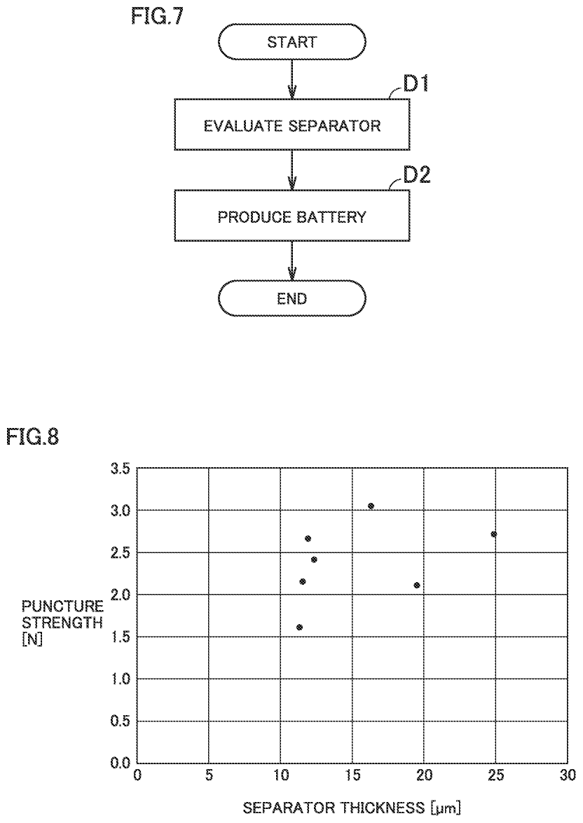

FIG. 7 is a schematic flowchart of a third production method.

The third production method is a production method for a battery. The third production method includes “(D1) evaluating a separator” and “(D2) producing a battery”.

### <<(D1) Evaluating Separator>>

The third production method includes evaluating separator 12 by the present evaluation method. Separator 12 may be prepared by any method. For example, a ready-made separator 12 may be obtained from the market. For example, separator 12 may be manufactured. By the present evaluation method, separator 12 is evaluated. For example, the magnitude of short circuit load may be considered to decide failure or no-failure of separator 12.

### <<(D2) Producing Battery>>

The third production method includes producing a battery including separator 12.

For example, a positive electrode, separator 12, and a negative electrode are prepared. For example, each of the positive electrode, separator 12, and the negative electrode may be a belt-shaped sheet. For example, the positive electrode, separator 12, and the negative electrode may be stacked to form a stack. Separator 12 is interposed between the positive electrode and the negative electrode. The stack may be wound in a spiral manner to form a wound-type electrode assembly. The electrode assembly may be shaped into a flat form.

For example, each of the positive electrode, separator 12, and the negative electrode may be in sheet form. For example, the positive electrode and the negative electrode may be stacked alternately with separator 12 interposed therebetween, to form a stack-type electrode assembly.

For example, the electrode assembly and an electrolyte solution may be encapsulated inside a casing to produce a battery. The battery includes separator 12 the short circuit load of which is already evaluated. The battery may have a short circuit resistance the level of which depends on the short circuit load of separator 12. For example, battery designing may be attempted by referring to the results of battery short circuit test and the short circuit load of separator 12.

### Examples

By first to fifth evaluation examples, the separator was evaluated. The present example includes fourth and fifth evaluation examples. The present example does not include first to third evaluation examples.

### <<Preparing Test Piece>>

A separator was prepared. The separator was a porous polyolefin film. The separator was produced by a dry process (a stretching method).

An electrode (a negative electrode) was prepared. The electrode had a thickness of 66 μm. The electrode included an active material layer and a current collector. The active material layer was placed on both sides of the current collector. The active material layer had a coating weight of 3.30 mg/cm² per side. The active material layer included graphite, CMC, and SBR. The current collector included a Cu foil.

In the first evaluation example, the separator alone was regarded as a test piece. In the second to fifth evaluation examples, the separator was placed on a surface of the electrode (the active material layer) to prepare a test piece.

### <<First Evaluation Example>>

In the first evaluation example, puncture strength (maximum force) of the separator alone was measured in accordance with “JIS Z 1707 General rules of plastic films for food packaging”.

FIG. 8 is a graph for the relationship between puncture strength and separator thickness in the first evaluation example. In the first evaluation example, the correlation between separator thickness and puncture strength tends to be weak. In the first evaluation example, the separator can extend in a sticking direction. A separator that extends easily seems to have a greater puncture strength. This may weaken the correlation between separator thickness and puncture strength.

Inside an actual battery, there would be little space for a separator to extend into. The puncture strength in the first evaluation example is not considered to be a suitable index for the strength of a separator inside an actual battery.

### <<Second Evaluation Example>>

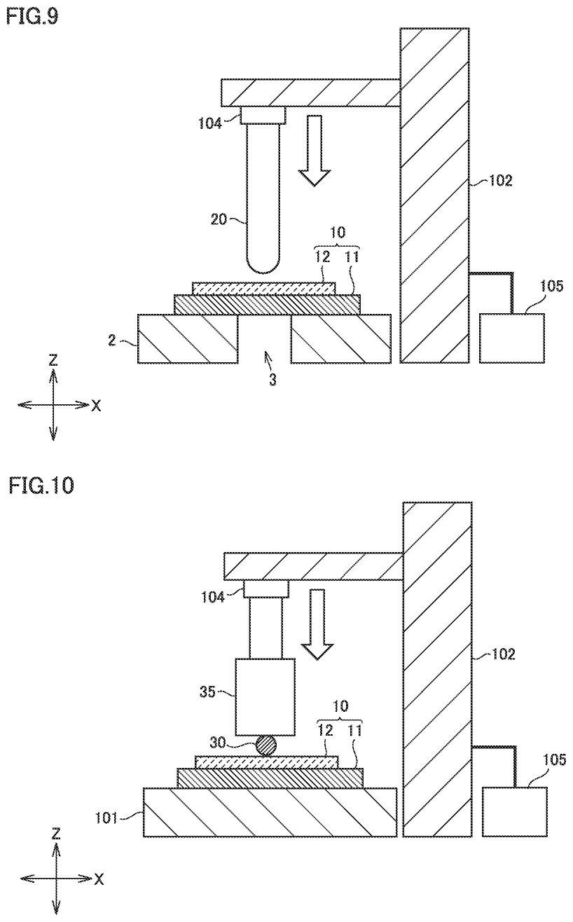

FIG. 9 is a conceptual view illustrating the second evaluation example.

Puncturing tool 20 was stuck into test piece 10 (separator 12). During sticking, the amount of displacement of puncturing tool 20 and the load were measured. By this, a stress-strain curve was obtained.

Extracting a peak for separator 12 from the stress-strain curve for test piece 10 was considered. The strength of separator 12 is smaller than that of substrate 11. Because of this, the peak for separator 12 was buried in the peak for substrate 11, making it difficult to extract the peak for separator 12. That is, it was difficult to evaluate the strength of separator 12 in the second evaluation example.

### <<Third Evaluation Example>>

FIG. 10 is a conceptual view illustrating the third evaluation example.

A simulant foreign object 30 was prepared. Simulant foreign object 30 was a Cu wire (diameter, 100 μm). Simulant foreign object 30 was placed on a surface of separator 12. Simulant foreign object 30 was pressed into separator 12 with the use of a pressing jig 35. During pressing, the amount of displacement of pressing jig 35 and the load were measured. By this, a stress-strain curve was obtained.

In the third evaluation example, measurement results varied widely. This variation may be attributed to the surface profile of simulant foreign object 30 (such as flash), inconsistent contact between simulant foreign object 30 and test piece 10, and the like.

### <<Fourth Evaluation Example>>

FIG. 11 is a schematic view illustrating a puncturing tool in the fourth evaluation example.

As a puncturing tool, a needle with a hemispherical tip was prepared. The tip radius (SR) of the needle was 0.5 mm. The diameter (φ) of the needle was 1 mm.

The fourth evaluation example was carried out with the use of the present evaluation system (see FIG. 2).

Test piece 10 (separator 12, substrate 11) was placed on stage 101. Stage 101 was electrically conductive. Puncturing tool 20 was attached to driver apparatus 102. As resistivity-measuring apparatus 103, a tester was prepared. The measurement range of the tester was from 419.9 Ω to 41.99 MΩ. Resistivity-measuring apparatus 103 was connected to stage 101 and puncturing tool 20. The displayed value on the tester at this point in time was infinity.

At the time when puncturing tool 20 was lowered to reach immediately above separator 12, the movement of puncturing tool 20 was paused. Then, puncturing tool 20 was lowered at a test velocity of 0.5 mm/min, and thereby puncturing tool 20 was stuck into separator 12.

At the time when the displayed value of the tester changed from infinity to 40 MΩ (that is, when the electrical resistance decreased to the short circuit resistance), puncturing tool 20 was stopped. The load at this time (short circuit load) was measured with load-measuring apparatus 104.

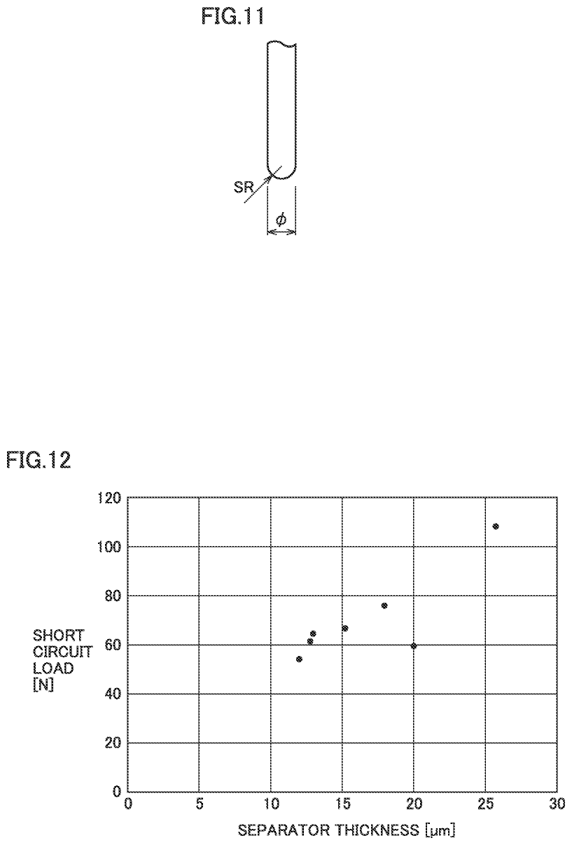

FIG. 12 is a graph for the relationship between short circuit load and separator thickness in the fourth evaluation example. The short circuit load correlates with the separator thickness. This may be because extension of the separator in a sticking direction is hindered. It is considered that the short circuit load reflects well the strength of the separator when a foreign object enters between electrodes within a battery.

### <<Fifth Evaluation Example>>

FIG. 13 is a schematic view illustrating a puncturing tool in the fifth evaluation example.

As a puncturing tool, a needle having a taper R tip shape was prepared. The tip radius (SR) of the needle was 0.1 mm. The diameter (φ) of the needle was 1 mm. The taper angle (θ) was 60°. The separator was evaluated in the same manner as in the fourth evaluation example except that the puncturing tool according to FIG. 13 was used.

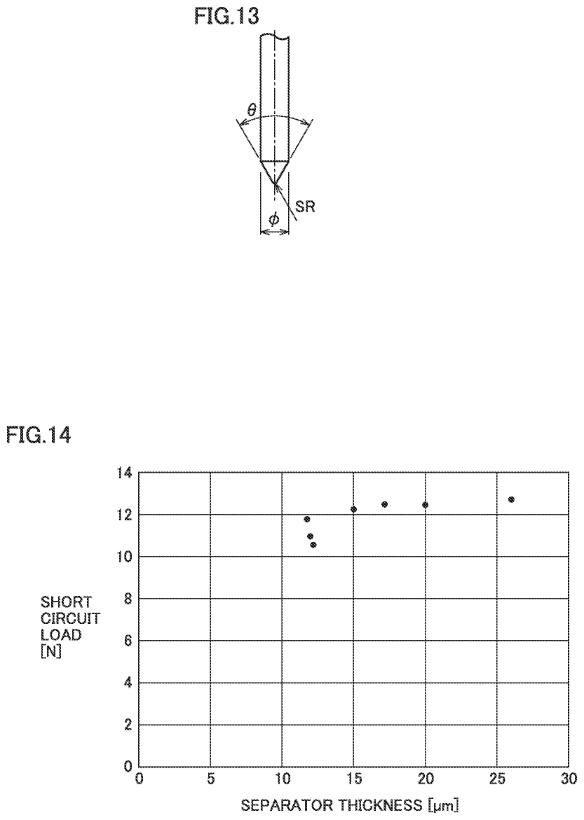

FIG. 14 is a graph for the relationship between short circuit load and separator thickness in the fifth evaluation example. In the fifth evaluation example (FIG. 14), the absolute value of short circuit load is lower than in the fourth evaluation example (FIG. 12). It may be because the evaluation conditions in the fifth evaluation example are harsher than those in the fourth evaluation example. In the fifth evaluation example, the tip of the puncturing tool is sharper than in the fourth evaluation example (see FIGS. 11 and 13). According to the present evaluation method, the shape of the puncturing tool may be adjusted to simulate various modes of failure within a battery.

The present embodiment and the present example are illustrative in any respect. The present embodiment and the present example are non-restrictive. The technical scope of the present disclosure encompasses any modifications within the meaning and the scope equivalent to the terms of the claims. For example, it is expected that certain configurations of the present embodiments and the present examples can be optionally combined.

What is claimed is:

1. An evaluation method for a separator for a battery, comprising:
(a) preparing a test piece by placing a separator on a surface of a substrate;
(b) sticking a puncturing tool into the separator, from a side of the test piece opposite to where the substrate is placed, in a thickness direction of the separator;
(c) measuring an electrical resistance between the puncturing tool and the substrate while the puncturing tool is being stuck into the separator; and
(d) evaluating the separator based on a magnitude of a load applied to the puncturing tool at the time when the electrical resistance has decreased to a predetermined value, wherein
each of the substrate and the puncturing tool is electrically conductive.

2. The evaluation method for a separator for a battery according to claim 1, further comprising:
(e) evaluating the separator based on an amount of displacement of the puncturing tool at the time when the electrical resistance has decreased to the predetermined value.

3. The evaluation method for a separator for a battery according to claim 1, wherein the substrate includes an electrode for a battery.

4. The evaluation method for a separator for a battery according to claim 3, wherein
the substrate includes an electrode assembly for a battery, and
the electrode assembly for a battery includes a plurality of the electrodes for a battery.

5. An evaluation system for implementing the evaluation method for a separator for a battery according to claim 1, the evaluation system comprising;
a stage;
a driver apparatus;
a resistivity-measuring apparatus; and
a load-measuring apparatus, wherein
the stage is to receive the test piece on itself,
the driver apparatus is to move the puncturing tool in the thickness direction of the separator toward the test piece on the stage,
the resistivity-measuring apparatus is to measure the electrical resistance between the puncturing tool and the substrate, and
the load-measuring apparatus is to measure the load applied to the puncturing tool.

6. The evaluation system according to claim 5, wherein
the evaluation system further comprises a displacement-measuring apparatus, and
the displacement-measuring apparatus is to measure an amount of displacement of the puncturing tool.

7. The evaluation system according to claim 5, wherein
the stage is electrically conductive, and
the resistivity-measuring apparatus is to measure an electrical resistance between the puncturing tool and the stage.

8. A production method for a separator for a battery, comprising:
(A1) producing a separator; and
(A2) evaluating the separator by the evaluation method for a separator for a battery according to claim 1.

9. A production method for an electrode unit, comprising:
(B1) producing an electrode unit by placing a separator on a surface of an electrode for a battery; and
(B2) evaluating the separator by the evaluation method for a separator for a battery according to claim 3, by using the electrode unit as a test piece.

10. The production method for an electrode unit according to claim 9, wherein the separator is bonded to the surface of the electrode for a battery.

11. A production method for a battery, comprising:
(C1) producing an electrode unit by the production method for an electrode unit according to claims 9; and
(C2) producing a battery including the electrode unit.

12. A production method for a batter, comprising:
(D1) evaluating a separator by the evaluation method for a separator for a battery according to claims 1; and
(D2) producing a battery including the separator.
